(19) United States
(12) Patent Application Publication
(10) Pub. No.: US 2024/0091105 A1
(43) Pub. Date: Mar. 21, 2024

(54) Reconfigurable Smart Package

(71) Applicant: QuantaEd, LLC, San Diego, CA (US)

(72) Inventor: Mehran MEHREGANY, San Diego, CA (US)

(21) Appl. No.: 18/525,370

(22) Filed: Nov. 30, 2023

Related U.S. Application Data
(63) Continuation of application No. 16/434,981, filed on Jun. 7, 2019.

(51) Int. Cl. A61J 7/00; A61J 1/03; G01V 8/20

(52) U.S. Cl. CPC A61J 7/0076; A61J 1/035; G01V 8/20; A61J 2200/30

(57) ABSTRACT

A smart case capable of monitoring blister cards having a range of formats is presented. The smart case includes a sensor system able to detect a change in the lidding film and/or content of a blister card substantially anywhere over the lateral extent of the blister card. In some embodiments, an adapter is included that enables any of a set of blister cards having different configurations (e.g., reservoir numbers, sizes, shapes, and/or inter-reservoir spacings) to be mounted in the same smart case. In some embodiments, the adapter includes walls that mitigate crosstalk between adjacent reservoirs and sensors. Sensor systems in accordance with the present disclosure provide at least one signal for each reservoir on each blister card of the set. In some embodiments, the sensor system has a density of detector elements that is at least equal to the highest density of reservoirs in any blister card of the set.

### CROSS REFERENCE TO RELATED APPLICATIONS

This application is a continuation of U.S. patent application Ser. No. 16/434,981 filed Jun. 7, 2019 (Attorney Docket: 3005-011US1), which is incorporated by reference as if set forth at length herein.

Furthermore, the following cases are also incorporated herein by reference:
i. U.S. patent application Ser. No. 16/156,603, filed Oct. 10, 2018 (Attorney Docket: 3005-008US1) (hereinafter referred to as “the '603 application”);
ii. U.S. patent application Ser. No. 14/879,874, filed Oct. 9, 2015 (Attorney Docket: 3005-002US1) (hereinafter referred to as “the '874 application”);
iii. U.S. patent application Ser. No. 15/170,121, filed Jun. 1, 2016 (Attorney Docket: 3005-002US2) (hereinafter referred to as “the '121 application”);
iv. U.S. patent application Ser. No. 15/223,779, filed Jul. 29, 2016 (Attorney Docket: 3005-004US1) (hereinafter referred to as “the '779 application”); and
v. U.S. patent application Ser. No. 16/333,021, filed Mar. 13, 2019 (Attorney Docket: 3005-006US1) (hereinafter referred to as “the '021 application”).

If there are any contradictions or inconsistencies in language between this application and any of the cases that have been incorporated by reference that might affect the interpretation of the claims in this case, the claims in this application should be interpreted to be consistent with the language in this application.

### TECHNICAL FIELD

The present disclosure relates to packaging in general, and, more particularly, to smart packaging.

### BACKGROUND

Health care expectations and expenditures are growing worldwide. There is a strong desire to find ways to improve care outcomes and reduce care cost. Improving medication adherence (i.e., dosing-regimen compliance) is a strategy that holds promise to improve outcomes and reduce care cost. The reason is that, to be effective, prescription and over-the-counter medicines (a.k.a., drugs) require good adherence to a specific dosing regimen—taking the right dosage, with the right frequency (dosing interval), at the right time (dosing window) for the prescribed duration.

Unfortunately, dosing-regimen noncompliance is common and leads to a costly problem in many ways, from driving up health care costs to financial losses to the pharmaceutical industry to serious negative human impacts. When a patient practices poor adherence, efficient recovery is less likely, which lengthens recovery duration and can necessitate additional interventions. As a result, treatment outcome is compromised and care cost is increased.

A major portion of health care cost is due to treatment of chronic diseases. Chronic diseases are illnesses that last more than three months. They cannot be prevented by vaccination, and do not disappear on their own or get cured by medication. As a result, treatment typically deals with the resulting symptoms to ease the discomfort of the patient. Examples of chronic diseases include cardiovascular disease, cancer, diabetes, epilepsy and seizer, obesity, depression, schizophrenia, asthma and chronic obstructive pulmonary disease (COPD) to name a few. Chronic diseases usually lead to comorbidities, e.g., an obese person develops hypertension, diabetes, etc.

Chronic diseases are prevalent, particularly among the aging population. Nearly half of Americans have at least one chronic disease, at least half of whom have two or more chronic diseases. As a result, chronic diseases account for ˜75% of the total health care expenditure in the United States, which was $3.5 trillion in 2017. Trends are generally similar across the world.

It is common that patients, particularly chronic-disease patients, are on more than one medication—some more than a dozen. Many such medications require multiple doses per day. By way of example, a patient on medications A, B, C, D, E and F has to take: (i) A and C four times a day, i.e., morning, noon, evening and night; (ii) B every other day; (iii) D and E morning and night; and (iv) F at noon. It is clear, therefore, that managing adherence to such dosing regimens is challenging. This challenge increases with number of medications and their respective dosing regimen variations.

One approach to improve adherence has been to package patient medications by dosage, with instructions of what dose to take when. One conventional packaging scheme employs blister cards, wherein each blister on the card contains the medications for each dosing window. These blister cards tend to be larger than blister cards for single medications because the blisters must be large enough to accommodate multiple medications per dose. In addition, such blister cards tend to be configured in a weekly format, e.g., an array of 7×4 blisters.

Another conventional approach to improving medication adherence employs “reminders,” which can be automated and delivered in the form of alarms, text message, calls and/or e-mails. Typically, a mobile device (usually a smart phone or tablet) running mobile-application software functions as a user interface for receiving such notifications. Alternatively, medication can be placed in a case/vessel/package that incorporates electronic provisions for tracking adherence and issuing audio/visual notifications. In some cases, the case/vessel/package is wirelessly linked to a mobile device and notifications are provided to the user via the mobile device, on the vessel/package, or both. Approaches such as the above are commonly referred to as “smart packaging”.

### SUMMARY

The present disclosure teaches systems and methods that facilitate tracking of adherence to medication-dosing regimens through connected, smart packaging. Smart cases in accordance with the present disclosure are particularly well suited for use with medicines provided in blister-cards, multi-medicine blister cards, and the like.

Like smart cases known in the prior art, smart cases in accordance with the present invention include a sensor system that can monitor the state of the lidding film and/or content in the reservoirs of a blister card; however, prior-art sensor systems are configured to monitor one particular blister card having a specific configuration (e.g., reservoir sizes, shapes, and/or inter-reservoir spacings).

In sharp contrast to the prior art, a smart case in accordance with the present disclosure includes a sensor system that can be used to monitor multiple blister cards that have different configurations. Embodiments in accordance with the present disclosure include a plurality of adapters, each of which can be mounted in the same smart case to enable it to accept any of a set of blister cards having different configurations. In some embodiments, the adapters are annular frames having a uniform outer shape and size and an inner annulus that is based on the lateral extent of a different blister card of the set. In some embodiments, some of the adapters include grids of walls that are arranged to surround each blister on its corresponding blister card of the set to securely locate and hold the blister card in the smart case. Furthermore, in some embodiments, the walls also serve to mitigate optical crosstalk between neighboring reservoirs and sensor elements.

An illustrative embodiment is a smart case that includes a housing that includes an electronics module and a detection module. The electronics module includes electronic circuitry for interfacing to a sensor system for monitoring a blister card and communications circuitry for communicating with an external device, such as a mobile phone, as well as other electronic functionality. The detection module comprises a receiver and the sensor system.

The receiver includes a seat, an adapter, and a frame, where the seat supports the adapter and the blister card, the adapter locates the blister card, and the frame secures the blister card and adapter against the seat. The adapter includes a grid of walls that define cells that substantially match the three-dimensional shape of the reservoirs of the blister card such that the blisters of the blister card removably “nest” in the cells. The walls also function as light barriers that mitigate optical crosstalk between adjacent reservoirs and sensor elements.

In the illustrative embodiment, the sensor system includes a plurality of photodiodes that are arranged in an array on a substrate that resides between the seat and the adapter. The photodiode array is configured such that at least one photodiode is located beneath each reservoir. As a result, when the lidding film of a reservoir is removed and its content is dispensed, its respective photodiode will detect light that passes through the reservoir and generate an output signal that indicates the content has been dispensed.

In some embodiments, a smart case includes a plurality of adapters, each having an inner annulus suitable for a different blister card of a set of blister cards. In some embodiments, each adapter includes a grid of walls and cells, where each arrangement matches the configuration of a different blister card of a set of blister cards. In some embodiments, the sensors system includes a photodiode array having a density that is equal to or greater than the highest reservoir density of the reservoirs of the set of blister cards for which the smart case is operative.

In some embodiments, an adapter comprises walls that include electrodes that define the sensor system, where the electrodes are operative for monitoring the content of each reservoir via electrical capacitance tomography.

In some embodiments, the sensor system includes a focal-plane array that can provide the position of any light incident upon it. As a result, the focal plane array is capable of monitoring the state of a blister card having any configuration.

In some embodiments, the sensor system includes a plurality of connectors that are electrically couplable with an electrically conductive lidding film of a blister card, where the connectors enable monitoring of the state of the lidding film via a tomographic imaging technique, such as electrical resistance tomography (ERT) and electrical impedance tomography (EIT).

An embodiment in accordance with the present disclosure is a smart case (500) for monitoring the state of a first blister card (122) comprising a first plurality of blisters (210) and a first lidding film (204) having a first plurality of dispensing regions (212), the first plurality of blisters (210) and first plurality of dispensing regions collectively defining a first plurality of reservoirs (206) that contain first content (208), wherein the first plurality of blisters collectively define a first blister region (218), and wherein the smart case comprises a detection module (106) that includes: a receiver (112) that is configured to locate and secure the first blister card, wherein the receiver is located in a first body portion (P2), and wherein the receiver includes a first adapter (508) having a flange (520) that has an outer perimeter (OP1) and a first inner perimeter (IP1), the outer perimeter being based on the dimensions of the first body portion and the inner perimeter being based on the first blister region; and a sensor system (114) that is configured to monitor the state of at least one of the first lidding film and the first content.

Another embodiment in accordance with the present disclosure is a smart case (500) for monitoring the state of any blister card of a set of blister cards (122), each blister card of the set thereof comprising a plurality of blisters (210) and a lidding film (204) having a plurality of dispensing regions (212), the plurality of blisters (210) and plurality of dispensing regions collectively defining a plurality of reservoirs (206) that contain first content (208), wherein the plurality of blisters collectively define a blister region (218), and each blister card of the set thereof having a different configuration, wherein the smart case comprises: a first body portion (P2) for holding a receiver (112) that is reconfigurable to locate and secure each blister card of the set thereof; the receiver; and a sensor system (114) that is operative for monitoring the state of each blister card of the set thereof.

### BRIEF DESCRIPTION OF THE DRAWINGS

FIG. 1 depicts a functional block diagram of a medicine case in accordance with the present disclosure.

FIG. 2A depicts a schematic drawing of a perspective view of blister card 122.

FIG. 2B depicts a schematic drawing of a sectional view of an exemplary reservoir of blister card 122.

FIGS. 2C-D depict plan view schematic drawings of the front (forming-film side) and back (lidding-film side) of blister card 122.

3A-B depict schematic drawings of perspective-top and plan-bottom views, respectively, of case 100.

FIG. 3C depicts a schematic drawing of an exploded sectional view of receiver 112, sensor system 114, and blister card 122.

FIG. 3D depicts a schematic drawing of top view of sensor system 114.

FIGS. 3E-F depict schematic drawings of examples of electronically actuatable latches suitable for use in accordance with the present disclosure.

FIG. 4A depicts a schematic drawing of a perspective top view of an alternative embodiment of a smart case in accordance with the present disclosure.

FIG. 4B depicts a schematic drawing of a sectional view of a portion of detection module 402.

FIG. 5A depicts a schematic drawing of a sectional view of another alternative detection module in accordance with the present disclosure.

FIG. 5B-C depict top and perspective views, respectively, of adapter 508.

FIGS. 5D-E depict schematic drawings of an alternative adapter in accordance with the present disclosure.

FIG. 6A depicts a schematic drawing of a sectional view of yet another alternative detection module in accordance with the present disclosure.

FIG. 6B depicts detection module 600 with the addition of optional cover 606.

FIG. 7A depicts a schematic drawing of an exploded perspective view of the salient features of yet another alternative smart case in accordance with the present disclosure.

FIGS. 7B-C depict schematic drawings of exploded perspective views of smart case 700 configured for use with blister cards that are larger and smaller, respectively, than blister card 122.

FIGS. 8A-B depict exploded schematic perspective drawings of two alternative receivers in accordance with the present disclosure.

### DETAILED DESCRIPTION

It should be noted that, although embodiments in accordance with the present disclosure are described herein with respect to medications as an exemplary application, concepts of the present disclosure are applicable to a wide range of other applications; therefore, the use of medication should not be viewed as limiting the scope of the utility of the concepts described herein. Other examples of applications in which the smart packaging concepts of the present disclosure can be used include, without limitation, other forms of medication, non-medication products (e.g., vitamins, supplements, etc.), foodstuff, gum, beverages, toiletry and beauty care products, razor blades, consumer electronics, toner cartridges, toys, tools, etc. In other words, the teachings of the present disclosure are applicable to a wide range of product-tracking applications.

Accordingly, for the purposes of this Specification, including the appended claims, the term “content” is used as a general term that encompasses all products packaged in blister-card format.

Furthermore, although the examples described herein are directed to monitoring the state of blister cards containing medicine in tablet form, many types of medication and non-medication (e.g., vitamins, supplements, breath freshener, etc.) are offered in forms other than that of a tablet but that are suitable for packaging in a blister card, such as capsules, lozenges, powders, gels, liquids, etc. For the purposes of this Specification, including the appended claims, the term “tablet” is used as a general term that encompasses all such medicinal and non-medicinal forms.

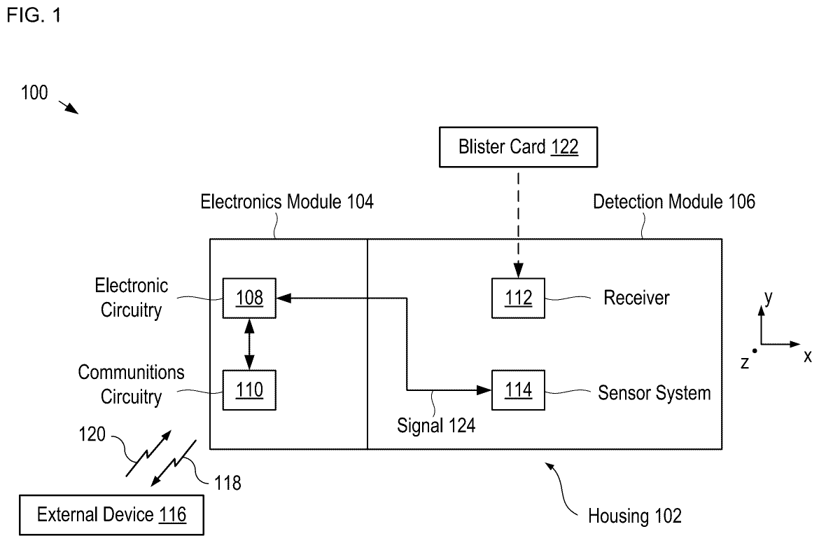

FIG. 1 depicts a functional block diagram of a medicine case in accordance with the present disclosure. Case 100 includes housing 102, electronics module 104, and detection module 106.

Housing 102 is a non-modular, molded plastic case having a first portion that contains electronics module 104 and a second portion that contains detection module 106. The second portion includes a body and a lid that can be opened and closed to enable insertion of blister card 122 and then control access to it. In the depicted example, the body and lid are connected along one edge via a flexible hinge portion. As discussed below, the body also includes an electronically actuatable clasp that engages a catch on the lid for controlling access to the blister card.

It should be noted that, although case 100 is a non-modular case that includes non-separable electronics and detection modules, the teachings of the present disclosure are applicable to smart cases having electronics and detection modules that are separable and the description of a non-modular case should not be considered limiting to the scope of the appended claims. Examples of modular smart cases in accordance with the present disclosure are described in the '021 application.

Electronics module 104 is a module that contains electronics circuitry 108 for interfacing to sensor system 114, as well as communications circuitry 110. Electronics module 104 is analogous to electronics modules described in detail in the '874, '121, '779, and '021 applications.

Electronic circuitry 108 enables the electronics module to receive and condition (e.g., provide pre-amplification, digitization, etc.) one or more signals from sensor system 114 of detection module 106, provide power conditioning and management, display information to the user, etc. In some embodiments, electronic circuitry 108 includes one or more of the following:
i. controllers/processors; or
ii. memory; or
iii. sensor circuitry; or
iv. power circuitry; or
v. display circuitry and/or one or more displays; or
vi. sleep-mode circuitry; or
vii. energy-storage systems; or
viii. clock circuitry; or
ix. energy-scavenging systems; or
x. sensor-interface circuitry; or
xi. on-case alerts (e.g., LCD displays, speakers, buzzers, etc.); or
xii. sensors, such as environmental (e.g., touch, temperature, acceleration, humidity, shock, geolocation, etc.), biosensors (e.g., bioimpedance, biopotential, breath analyzer, alcohol detector, etc.), and the like; or
xiii. any combination of i, ii, iii, iv, v, vi, vii, viii, ix, x, xi, and xii.

Communications circuitry 110 enables communication with external device 116. In the depicted example, communications circuitry 110 includes a wireless Bluetooth Low-Energy (BLE) transceiver for transmitting operational communications 120 and receiving status signal 118 to/from external device 116. In some embodiments, communications circuitry includes a different wired and/or wireless communications electronics, such as FireWire, USB, lightning connector, a dock connector, cellular, WiFi, near-field-communications (NFC) radio, optical links, etc.

Detection module 106 comprises receiver 112 and sensor system 114. Receiver 112 includes fixturing that is configured to position and hold blister card 122 in a location that enables its state to be monitored by sensor system 114. Detection module 106 is described in detail below.

In the depicted example, external device 116 is a smart phone that communicates with electronics module 104 via communications circuitry 110 and runs a software application (i.e., a mobile app) that provides assistance to the patient and/or caregiver to achieve and maintain good adherence to the prescribed drug regimen. In some embodiments, external device 116 is a different device, such as a different mobile device (e.g., smart watch, computer tablet, etc.), a computer, a gateway and/or a base station. Preferably, the software application running on external device 116 is supported by data analytics, which include artificial intelligence and machine learning. In some embodiments, external device 116 is connected to the cloud backend through wireless and/or wired communications.

The smart case and smartphone app determine the state of the blister card and provide a visual and/or audible indication of adherence. If failure to follow the regimen is detected, the smartphone contacts one or more people in the user's defined support group (e.g., a caregiver, spouse, child, doctor, etc.) to alert them that the user might require assistance.

In the depicted example, case 100 is a smart case for tracking the state of blister card 122, which contains medicinal content for a one-week medicinal regimen having four dosing periods (A through D) for each of seven days (1 through 7). In physical form, smart case 100 is analogous to those described in detail in the '874, '121, '779, and '021 applications. It should be noted that, while the depicted example is configured to monitor the state of a blister card having a 7×4 dosing arrangement, the principles of the present disclosure can be applied to a smart case suitable for use with any practical blister card arrangement and dosing schedule. For example, a 7×4 blister card may correspond to a single daily dose over 4 weeks, and a 4×1 blister card may correspond to a single daily dose over 4 days or a weekly dose over 4 weeks. Furthermore, the geometric placement of blister reservoirs may vary depending on human factors related to different use cases.

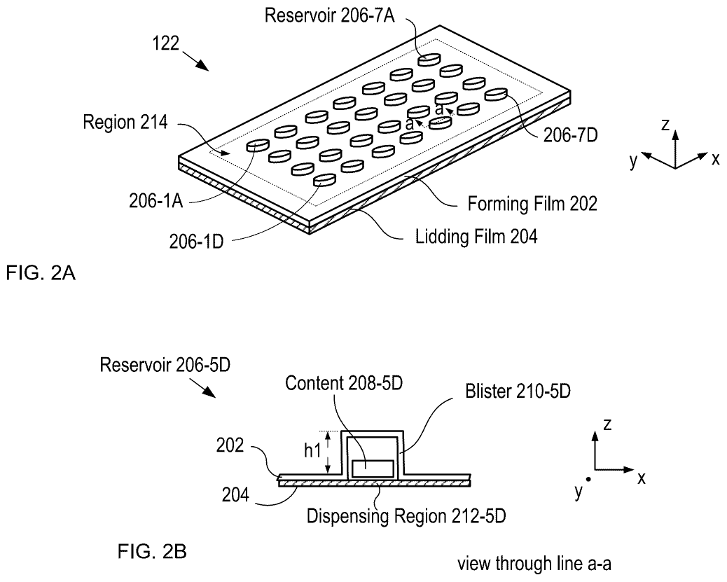

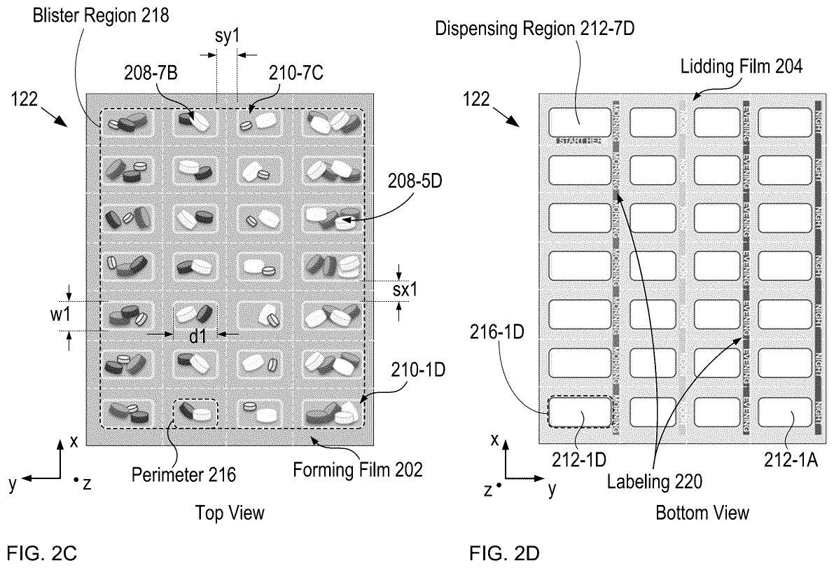

FIG. 2A depicts a schematic drawing of a perspective view of blister card 122.

FIG. 2B depicts a schematic drawing of a sectional view of an exemplary reservoir of blister card 122. The view depicted in FIG. 2B shows a cross-sectional view of reservoir 206-5D, which is taken through line a-a as shown in FIG. 2A. Reservoir 206-5D is representative of each of reservoirs 206-1A through 206-7D.

FIGS. 2C-D depict plan view schematic drawings of the front (forming-film side) and back (lidding-film side) of blister card 122.

Blister card 122 includes forming film 202 and lidding film 204, which are configured to define reservoirs 206-1A through 206-7D. Reservoirs 206-1A through 206-7D contain content 208-1A through 208-7D, respectively, each of which is the proper dose for a specific dosing window of a one-week medicinal regimen. In the depicted example, reservoirs 206-1A through 206-7D (referred to, collectively, as “reservoirs 206”) are arranged in a seven-row, four-column format.

Forming film 202 is a layer of thermoformed plastic in which cavities for holding medicinal content are formed, thereby defining blisters 210-1A through 210-7D (referred to, collectively, as blisters 210).

In the depicted example, lidding film 204 is a thin sheet of aluminum foil. In some embodiments, lidding film 204 is a sheet of different electrically conductive material. In some embodiments, lidding film 204 includes a sheet of electrically conductive material and a sheet of electrically insulating material, such as a paper sheet (with a printed calendar or instructions), polymer, etc. on the bottom side of the lidding film and/or sandwiched between the lidding and forming films. In some embodiments, lidding film 204 is a sheet of electrically non-conductive material (e.g., paper, etc.) or a laminate of electrically non-conductive materials (e.g., paper and polymer, etc.).

After content 208-1A through 208-7D (referred to, collectively, as content 208) is dispensed into blisters 210, lidding film 204 is joined with forming film 202 to seal the cavities, thereby completing reservoirs 206 and enclosing content 208. The portions of lidding film 204 under blisters 210-1A through 210-7D define dispensing regions 212-1A through 212-7D, respectively, through which content 208-1A through 208-7D is dispensed. Dispensing regions 212-1A through 212-7D (referred to, collectively, as dispensing regions 212) are all located within two-dimensional region 214. Each dispensing region 212 is characterized by perimeter 216, which is defined by the annulus at which its respective blister 210 is joined with lidding film 204.

Each of blisters 210 is characterized by a width, w1, in the x-direction, a depth, d1, in the y-direction, and a height, h1, in the z-direction. Blisters 210 are spaced apart by inter-reservoir spacings sx1 and sy1 in the x- and y-directions, respectively. For multi-medicine blister cards, at least one of the width, depth, height, and spacings of blisters 210 can be non-uniform over the blister card.

Perimeters 216 collectively define blister region 218, which is a two-dimensional region whose lateral extent is defined by the outer edges of the perimeters 216 of the outer rows and columns of blisters 210.

Blister card 122 also includes labeling 220 for providing dosing regimen information directly on the back of the blister card. In some cases, labeling 220 includes other information, such as a calendar that denotes the drug regimen, manufacturing information (date, lot number, etc.), and the like.

In some embodiments, case 100 is configured to monitor the state of a blister card in which at least one reservoir contains content other than a plurality of tablets. In some embodiments, some reservoirs may be left empty, for example, to simply maintain a habitual regimen routine or accommodate dosing schedules that do not use all of the reservoirs of a given blister card format. In some embodiments, one or more of such empty reservoirs may not have an intact lidding film or a lidding at all.

As will be apparent to one skilled in the art, at least some of reservoirs 206 are larger than those of single-medication blister cards to enable them to hold medicinal content for doses that include multiple tablets. An exemplary multi-tablet reservoir has a volume of 2 cubic inches, with a dispensing region 212 of approximately 1×2 inches and a blister height of approximately 1 inch. The spacing between reservoirs 206 (i.e., the inter-reservoir spacing of blister card 122) in each of the x- and y-dimensions is ¼ inch. As a result, a 7×4 format blister card size populated with such reservoirs requires that region 214 be about 10×10 inches.

It is an aspect of the present disclosure that the teachings herein enabling monitoring of any blister card.

One skilled in the art will recognize, after reading this Specification, that the design features of smart case 100 described herein are based on the particular arrangement of blister card 122, as well as the sensing technology used to monitor its state and, as a result, are merely exemplary. Myriad alternative design features are possible within the scope of the present disclosure.

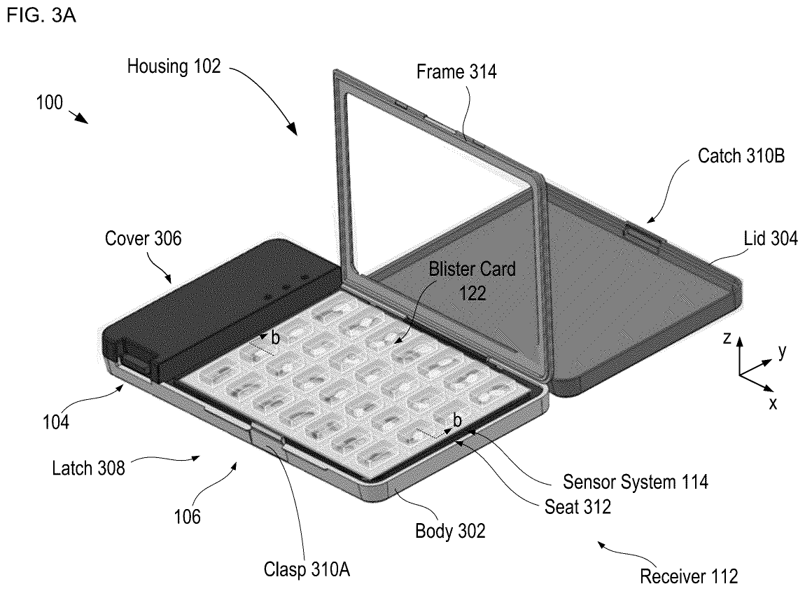

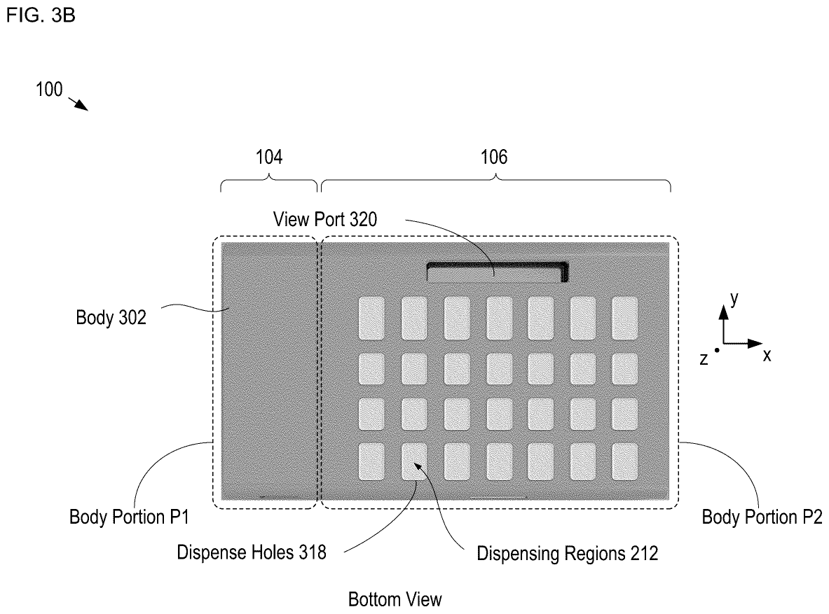

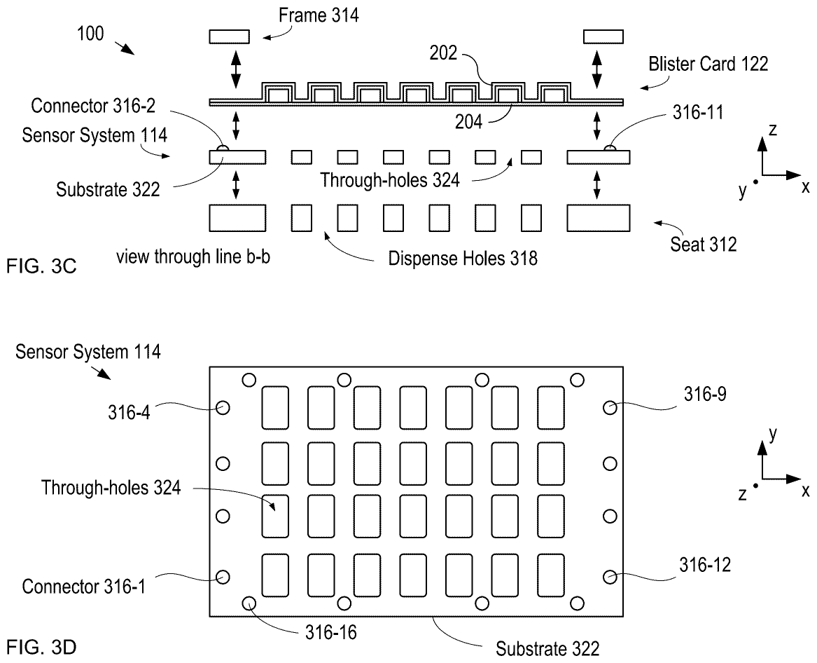

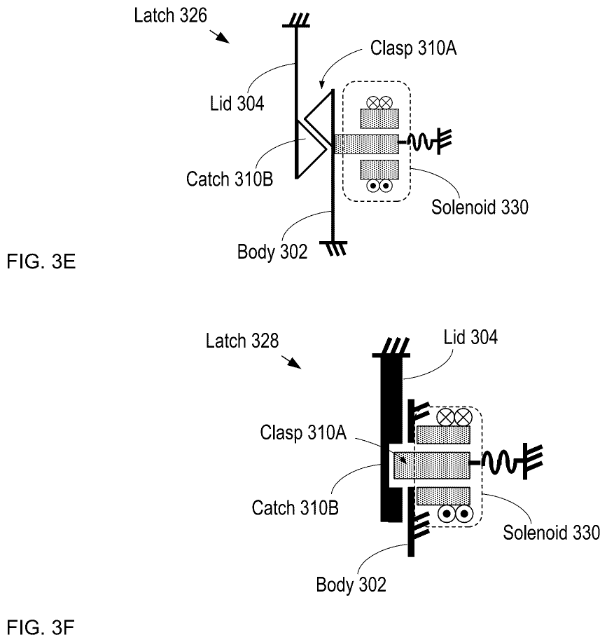

FIGS. 3A-B depict schematic drawings of perspective-top and plan-bottom views, respectively, of case 100.

Housing 102 includes body 302, lid 304, and cover 306. Body 302 extends along the entire bottom side of case 100. Cover 306 is rigidly affixed to body 302 to enclose and protect the components of electronics module 104, while lid 304 is a movable “clamshell” cover that is attached to body 302 via a hinge (not shown) along the length of detection module 106. As a result, lid 304 can be opened to enable access to content 208 and closed to protect blister card 122.

Body 302 includes body portions P1 and P2, which hold electronics module 104 and detection module 106, respectively. Body portion P2 includes dispense holes 318 and view port 320.

In the depicted example, since case 100 is non-modular, body portions P1 and P2 are contiguous sections of unitary body 302. As will be apparent to one skilled in the art, after reading this disclosure, for embodiments wherein a smart case is modular, body portions P1 and P2 are typically separate elements that can be reversibly connected as described in '021 application.

Dispense holes 318 are configured to enable passage of their respective content 208 through body portion P2 when dispensed.

View port 320 is an opening in body portion P2 that is configured to expose a portion of blister card 122 for viewing. View port 320 is optionally included to enable information printed on a blister card (such as a bar code, etc.) to be read through body 302.

In the depicted example, housing 102 includes latch 308, which is configured to reversibly secure lid 304 and body 302 in a closed configuration. Latch 308 comprises clasp 310A disposed on body 302 and catch 310B disposed on lid 304. To unlock latch 308 and enable lid 304 to move relative to body 302, the user mechanical decouples clasp 310A and catch 310B. A wide range of conventional mechanical latching designs can be used for latch 308, including passive designs based on magnets, zippers, Velcro, sticky films, etc.

In the depicted example, receiver 112 includes seat 312 and frame 314.

Seat 312 is the bottom portion of body 302 and is configured to accept sensor system 114 and blister card 122. Seat 312 includes dispense holes 318, which have a one-to-one correspondence with reservoirs 206.

Frame 314 is a rigid annular element that includes a large central opening configured to expose all of reservoirs 206 when blister card 122 is installed in case 100. Frame 314 is rotatable about a hinge (not shown) such that it can be closed over blister card 122 and latched in place. Once frame 314 is closed and latched, the blister card is securely held in an operatively coupled arrangement with sensor system 114, which is disposed on seat 312. In some embodiments, frame 314 includes a plurality of openings, each of which is configured to expose one or more reservoirs. In some embodiments, frame 314 is separately placed over the blister card after the blister card is inserted. In such embodiments, frame 314 is typically reversibly affixed in place by mechanical, magnets, electromagnets, latches and the like.

FIG. 3C depicts a schematic drawing of an exploded sectional view of receiver 112, sensor system 114, and blister card 122. The view depicted in FIG. 3C is taken through line b-b shown in FIG. 3A.

FIG. 3D depicts a schematic drawing of top view of sensor system 114. Sensor system 114 includes substrate 322, through-holes 324, and connectors 316-1 through 316-16.

Sensor system 114 includes connectors 316-1 through 316-16, which are configured to electrically couple with lidding film 204 and provide signal 124 to electronic circuitry 108. Sensor system 114 enables monitoring of the state of lidding film 204 via electrical resistance tomography (ERT) imaging, which is typically performed by electronic circuitry 108. As will be appreciated by one skilled in the art and as discussed in the '603 application, ERT imaging of lidding film 204 can be used to detect that a hole has been formed in a dispensing region 212 and where the hole is located. As a result, sensor system 114 can determine when one of content 208-1A through 208-7D has been dispensed and identify which dose was dispensed. In some embodiments, a different tomographic imaging technique is used (such as electrical impedance tomography (EIT), electrical capacitance tomography (ECT), and the like) to accommodate lidding films that are not electrically conductive.

Each of connectors 316-1 through 316-16 (referred to, collectively, as connectors 316) is a contact bump disposed on the top surface of substrate 322 and configured to enable blister card 122 to be operatively coupled with sensor system 114 without significant deformation of the blister card. Connectors 316 are electrically coupled with electronics module 104 via conventional electrical traces (not shown) disposed on substrate 322.

In some embodiments, connectors 316 are other than contact bumps, such as leaf-spring “wiper” contacts, “zero-insertion-force” (ZIF) connectors that close over the blister card after it has been inserted, and the like. In some embodiments, connectors 316 are configured to contact an electrically conductive lidding film capacitively (e.g., from the lidding film side, through an insulating layer disposed on the bottom side of lidding film 204, through the forming film when connectors 316 are placed on the forming-film side of the blister card 122, etc.). One skilled in the art will recognize, after reading this Specification, that the geometrical shapes and distributions of such resistive or capacitive contacts can be varied substantially to meet a wide range of performance specifications.

Substrate 322 is a printed-circuit board (PCB) that incorporates through-holes 324, which are configured to enable the passage of content 208 through the substrate 322 when the content is dispensed. It should be noted that, although the depicted example comprises a substrate 322 that is a PCB, myriad alternative materials can be used for substrate 322 without departing from the scope of the present invention.

Although in the illustrative embodiment sensor system 114 is an optical-sensor-based system, in some embodiments, sensor system 114 is based on a different sensing technology, wherein a plurality of sensors (e.g., capacitive sensors, strain sensors, optical sensors, acoustic sensors, tactile sensors, resistance sensors, impedance sensors, thermal sensors, magnetic sensors, etc.) having a one-to-one correspondence with dispensing regions 212-1A through 212-7D are employed. Examples of sensors suitable for use in such sensing schemes are described in detail in the '874, '121, '779, and '021 applications.

It is well understood that some medications, such as opioids, are highly addicting. It is an aspect of the present disclosure that access to such medication can be controlled via a smart case that includes an electrically-actuated latch that enables a dosing regimen to be programmed such that the smart case can only be opened during a dosing window. Such embodiments offer the promise of mitigating medication abuse by the user. Furthermore, if a user removes more doses than called for during an approved dosing window, it can be detected by case 100 (including if the blister card is completely removed) and an alert can be immediately issued to an appropriate party.

FIGS. 3E-F depict schematic drawings of examples of electronically actuatable latches suitable for use in accordance with the present disclosure.

Each of latches 326 and 328 comprises clasp 310A, catch 3106, and solenoid 330.

Solenoid 330 is a conventional solenoid that is mechanically coupled with clasp 310A. Solenoid 330 is configured to disengage clasp 310A from catch 310B only in response to a command from electronics module 104, thereby unlocking body 302 from lid 304 to enable access to content 208. In some embodiments, latch 308 can only be actuated for an authorized user (as determined via a user-entered access code, fingerprint, audio or image identification, etc.) and/or only during an appropriate dosing window. In such embodiments, case 100 provides a measure of protection against an accidental ingestion of content 208 (e.g., by a child, etc.), overmedication or abuse by the user, and the like.

In some applications, pushing content contained in a reservoir through its respective dispensing region and dispense hole is potentially problematic. For example, in the depicted example, one or more tablets might break when forced against one another as content 208 is dispensed from multi-tablet reservoir 206. The potential for tablet breakage increases with the number of tablets within the reservoir—particularly as the height and/or volume of the reservoir increases to accommodate more tablets and/or tablets are themselves fragile. Tablet breakage is also an issue for large disk-shaped tablets—even when contained in individual, conformal reservoirs.

As a result, in some cases, it is preferred that the content of a reservoir can be accessed by removal of at least a portion of its respective dispensing region instead of pushing the content through it. In such cases, blister card 122 is typically held in case 100 such that its lidding film faces toward the user. Once a sufficient portion of a dispensing region 212 is removed, the user can remove its respective content manually or turn the blister card over to enable the tablets to drop out of reservoir 206.

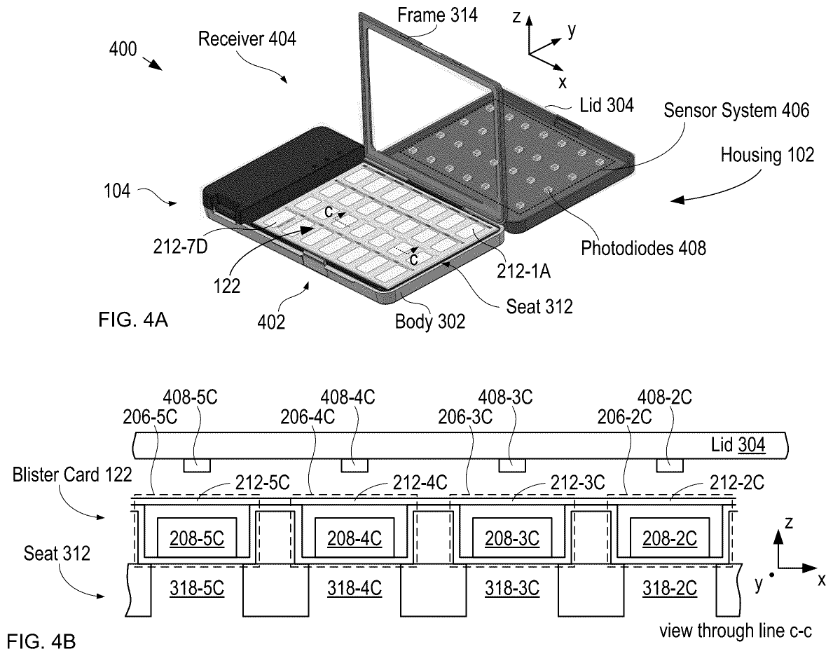

FIG. 4A depicts a schematic drawing of a perspective top view of an alternative embodiment of a smart case in accordance with the present disclosure.

Case 400 includes housing 102, electronics module 104, and detection module 402. Case 400 is analogous to case 100; however, case 400 is configured to monitor a blister card that is held with its lidding film facing lid 304, includes a sensor system that has a one-to-one correspondence with reservoirs 206, and such that the sensor system is disposed on the interior surface of lid 304.

FIG. 4B depicts a schematic drawing of a sectional view of a portion of detection module 402. The sectional view shown in FIG. 4B is taken through line c-c provided in FIG. 4A. Detection module 402 includes receiver 404 and sensor system 406.

Receiver 404 includes seat 312 and frame 314. Receiver 404 is analogous to receiver 112; however, receiver 404 is configured to position blister card 122 in an upside-down orientation (i.e., lidding film 204 is distal to seat 312) and such forming film 202 is disposed directly on seat 312. As a result, reservoirs 206 are aligned with dispense holes 318, which serve as viewing holes that enable the user to see content 208. In some embodiments, dispense holes 318 facilitate registration of the lateral position of blister card 122 by capturing a portion of its blisters.

For operation with a blister card whose lidding film includes printed instructions, it is preferable that frame 314 is optically transparent, or incorporates openings, to expose the information.

Sensing system 406 comprises photodiodes 408-1A through 408-7D (referred to, collectively, as photodiodes 408). Photodiodes 408 have a one-to-one correspondence to reservoirs 206-1A through 206-7D and are disposed on the interior surface of lid 304. Sensor system 406 also includes conventional electrical connections from photodiodes 408 to enable them to provide signal 124 to electronics module 104 (not shown), such as flexible ribbon cables that pass through the hinge cavity.

In operation, case 400 is opened to provide access to dispensing regions 212, enabling the user to remove the proper content from blister card 122 by removing at least some of the dispensing region from its reservoir.

Once the proper dose has been dispensed, lid 304 is closed. Since the lidding film material in the dispensing regions of dispensed content is missing, light can pass through these reservoirs to be detected by those photodiodes 408 aligned with them. These photodiodes provide output signals to electronics module 104, which determines whether a change in the output-signal configuration has occurred, thus indicating which content was dispensed. Since reservoirs whose dispensing regions are still intact will substantially block light, their respective photodiodes generate substantially no output signal.

When the light incident on a photodiode 408 is at a maximum, its respective reservoir is determined to be empty. When no light is incident on a photodiode 408, its respective reservoir is determined to be untouched (i.e., full). In some cases, however, a partial dose is left in a reservoir after its dispensing region has been opened. This affects the amount of light incident on its respective photodiode 408, thereby providing an indication of an incomplete dosing event. In addition, a sensor system that can detect the magnitude of light received by a photodiode enables monitoring of a blister card whose lidding film is optically transparent (e.g., a thin film of nylon, clear plastic, etc.). In some embodiments, machine learning and artificial intelligence enables a light signal whose intensity is somewhere between zero and the maximum to be related to the amount of content remaining in a reservoir (e.g., tablet types, remaining quantity, etc.).

In the depicted example, the light that passes through dispense holes 318 is ambient light. In some embodiments, one or more light sources are included in case 400 (e.g., disposed on seat 312) to provide the light detected by photodiodes 408. In some embodiments, each reservoir is provided a different light source. In such embodiments, the inclusion of dispense holes 318 in body 302 is optional.

It should be noted that, while sensing system 406 includes sensors that employ optical-sensing technology, cases in accordance with the present disclosure can employ sensing systems that are based on any of a wide range of sensing technologies. Sensing technologies that can be employed in embodiments in accordance with the present disclosure include, without limitation, capacitive sensing, strain sensing, optical sensing, acoustic sensing, tactile sensing, resistance sensing, impedance sensing, thermal sensing, magnetic sensing, and tomographic imaging. Examples of alternative sensing approaches suitable for use in accordance with the present disclosure are described in detail in the '874, '121, '779, and '021 applications.

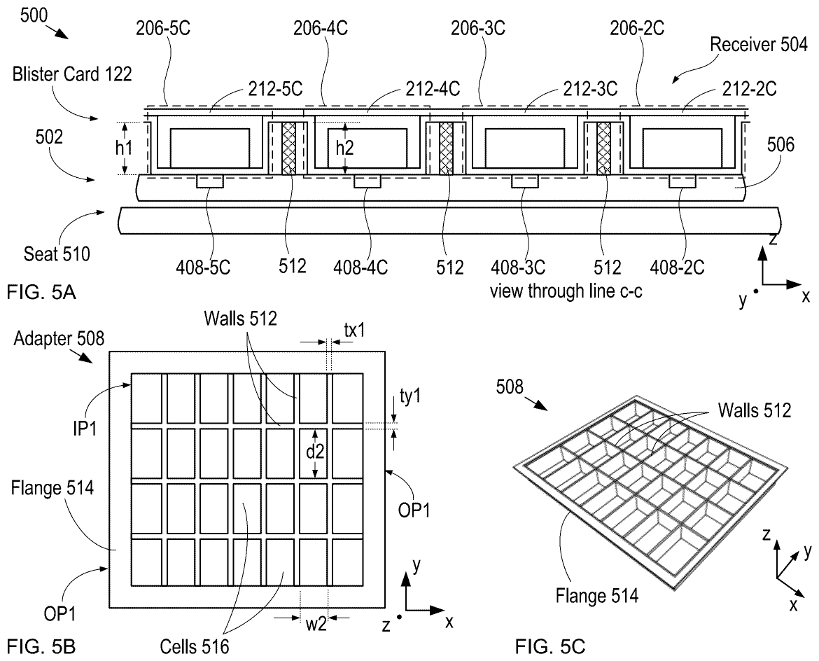

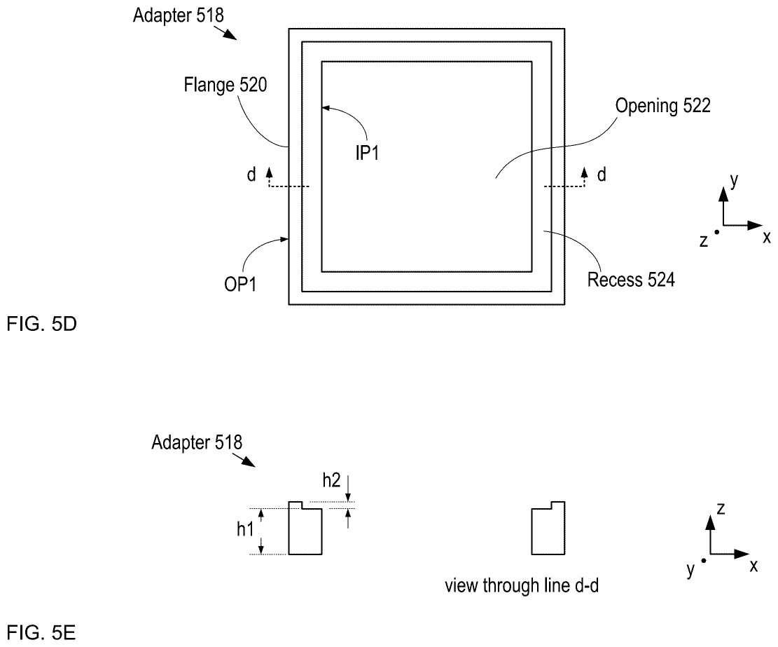

FIG. 5A depicts a schematic drawing of a sectional view of another alternative detection module in accordance with the present disclosure. The sectional view shown in FIG. 5A is analogous to the sectional view shown in FIG. 4B. Detection module 500 includes sensor system 502 and receiver 504. Detection module 500 is analogous to detection module 402; however, in detection module 500, sensor system 502 is a photodiode-based sensor system that is disposed underneath blister card 122. In addition, receiver 504 includes optional adapter 508, which includes a grid of walls that function as light-blocking features between adjacent reservoirs 206.

Sensor system 502 is analogous to sensor system 406; however, in sensor system 502, photodiodes 408 are located on substrate 506.

Substrate 506 is a PCB on which photodiodes 408 are mounted. In some embodiments, substrate 506 is another suitable substrate on which photodiodes 408 are disposed. In some embodiments, photodiodes 408 are monolithically integrated on or in substrate 506. In some embodiments, substrate 506 is an organic or an inorganic semiconductor facilitating the fabrication of photodiodes 408.

Sensor system 502 is configured to interrogate the lidding film of blister card 122 while lid 304 is open by virtue of photodiodes 408, which detect ambient light that passes through reservoirs 206 whose dispensing regions 212 have been removed. In some embodiments, however, one or more light sources are disposed on the interior surface of lid 304 to provide light that can be detected by photodiodes 408 when the lid is closed.

Receiver 504 includes seat 510, frame 314 (not shown), and optional adapter 508.

Seat 510 mechanically supports sensor system 502, adapter 508, and blister card 122. In the depicted example, seat 510 is the bottom of body 302. It should be noted that, since content 208 is dispensed from the top side of detection module 500, seat 510 does not require dispense holes 318.

In some instances, light can leak from an opened reservoir to the photodiode of a neighboring reservoir (referred to as “optical crosstalk”), which can cause problems. For example, optical crosstalk can lead to confusion regarding which of content 208-1A through 208-7D has been dispensed, or corrupt the analysis used to determine the amount and type of content remaining in a partially empty reservoir.

In the depicted example, therefore, the inclusion of optional adapter 508 in receiver 504 affords significant advantages over prior-art smart cases. First, the walls of the adapter substantially block light from passing between adjacent reservoirs of a blister card, thereby mitigating optical crosstalk between neighboring reservoirs and photodiodes. Second, as discussed below, adapters can enable the same smart case to accommodate a wide range of blister cards having different configurations. In some embodiments, adapter 508 is not included.

FIG. 5B-C depict top and perspective views, respectively, of adapter 508. Adapter 508 comprises walls 512 and flange 514. In the depicted example, adapter 508 is made of injection-molded plastic and is substantially optically opaque. In some embodiments, adapter 508 is made of a different suitable opaque material (e.g., metal, ceramic, etc.).

Adapter 508 comprises a grid of orthogonal walls 512 having height, h1, which is equal to the height, h1, of blisters 210 so that adapter 508 provides mechanical support for blister card 122. Walls 512 collectively define cells 516, which are characterized by a width, w2, in the x-direction, a depth, d2, in the y-direction, and a height, h2, in the z-direction. Typically, at least one of the dimensions of cell 516 is based on a dimension of blister 210. In the depicted example, cells 516 have substantially the same three-dimensional shape as blisters 210; therefore, all three dimensions of the cells match those of the blisters (i.e., w2=w1, d2=d1, and h2=h1). As a result, cells 516 capture blisters 210, thereby restricting the lateral movement of blister card 122.

Flange 514 is an annular plate that extends outward from walls 512 at the top of adapter 508. Flange 514 has outer perimeter OP1 and inner perimeter IP1. The value of OP1 is selected based on the dimensions of body portion P2 and, typically, is selected to enable adapter 508 to fit snugly within body portion P2. The value of IP1 is selected to enable all of blisters 210 of blister card 122 to fit within the flange. In the depicted example, IP1 is configured to match the perimeter of blister region 218. In some embodiments, IP1 is configured to be larger than the perimeter of blister region 218, as discussed above and with respect to FIG. 2C.

In the depicted example, adapter 508 is integral (i.e., non-removable) to detection module 500; however, in some embodiments the adapter is removable such that different adapters can be used with the same smart case, thereby enabling the case to be used with a set of blister cards having different configurations. Adapter 508 can be removably secured in the detection module using a variety of latching schemes (snapping, sliding, locking, magnetic, electromagnetic, etc.).

It should be noted that walls 512 of adapter 508 are substantially straight and vertical and have thicknesses, tx and ty, in the x- and y-directions, respectively, that are based on the inter-reservoir spacings sx1 and sy1 between blisters 210. In some embodiments, walls 512 have a shape that affords additional functionality, however.

FIGS. 5D-E depict schematic drawings of an alternative adapter in accordance with the present disclosure. Adapter 518 is an annular element having flange 520 and opening 522.

Flange 520 is analogous to flange 514 and has an outer perimeter OP1 that enables it to fit snugly within body portion P2.

Opening 522 has an inner perimeter IP1 that substantially matches the perimeter of blister region 218. As a result, blisters 210 of blister card 122 fits snugly into opening 522, thereby mitigating lateral movement of the blister card relative to flange 520. In some embodiments, opening 522 is larger in at least one dimension than blister region 218.

Flange 520 also includes optional recess 524, which has a perimeter that substantially matches the perimeter of blister card 122 and a height, h2, that is equal to the combined thickness of forming film 202 and lidding film 204. As a result, the blister card sits snugly within recess 524 even when opening 522 is larger than blister region 218.

In the depicted example, the height of recess 524 above the bottom of flange 520 is equal to the height, h1, of blisters 210. In some embodiments, however, recess 524 resides at a different height above the bottom of flange 520.

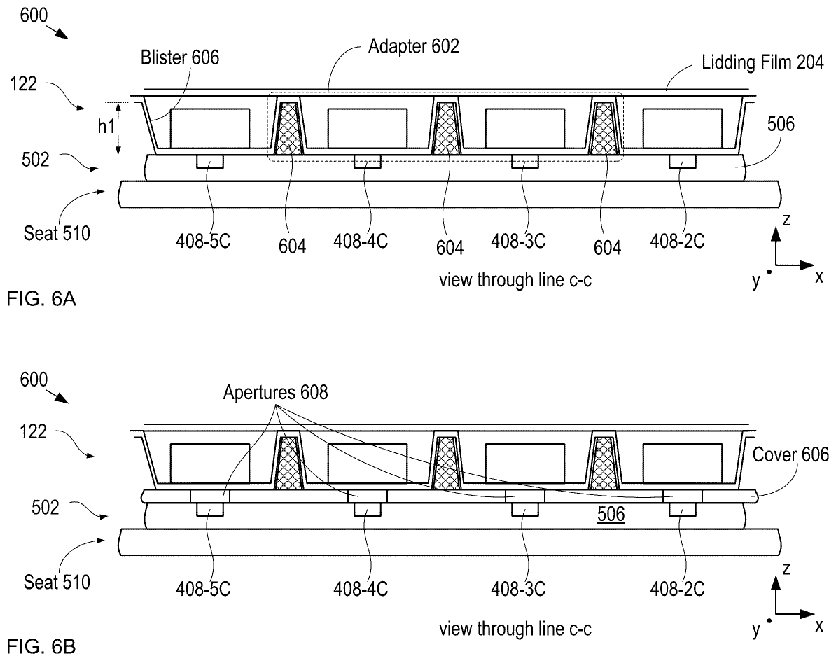

FIG. 6A depicts a schematic drawing of a sectional view of yet another alternative detection module in accordance with the present disclosure. Detection module 600 is analogous to detection module 500; however, detection module 600 includes an adapter whose walls conform to the shape of the blisters of the blister card for which it is intended. Adapter 602 includes flange 514 and walls 604.

Walls 604 of adapter 602 are tapered to conform to the shape of blisters 210, which have sidewalls that taper inward from lidding film 204 (i.e., the top of the blister is narrower than its base where it meets the lidding film). In others words, walls 602 define cells that intimately surround blisters 210. As a result, the large contact area between the blisters 210 and walls 604 enable conformal walls 604 to better “grip” the blister card and keep it in its desired location. The grip between the walls and blisters can be further enhanced by walls having a tacky surface.

It should be noted that walls 512 and 604 represent just two exemplary shapes of adapter walls that are within the scope of the present disclosure. In some embodiments, the shape of the walls is dictated by the additional functionality desired for an adapter.

In some embodiments, an adapter incorporates electrical functionality, such as indicator lights, electrodes/elements for other types of sensing (e.g., capacitive, acoustic, optical, magnetic, tactile, etc.), etc. For example, in some embodiments adapter walls include capacitive electrodes that enable imaging of the content of the reservoirs via electrical impendence tomography, as described in the '121 application, or by capacitive sensing schemes described in the '779 application. Electrical connection to electrical devices disposed on the walls of an adapter can be established via, for example, electrical connectors, proximity contacts, and the like.

It should be noted that, in some embodiments, the height of the walls of an adapter is selected based on their desired functionality beyond their light-blocking capability. For example, although walls 512 and 604 have height h1 (i.e., the same height as blisters 210), the walls can be shorter or longer than the height of the blisters to control the vertical position of blister card 122, secure the blister card in place, and/or optimize sensing capability. For example, in embodiments in which walls 512 and/or 604 include electrodes to effect capacitive sensing, a wall height that locates the bottom of the blisters in close proximity to their respective capacitive sensors can be beneficial.

In some embodiments, not all of the electronic circuitry of a smart case is located in electronics module 104. Instead, in some embodiments, substrate 506 (or seat 312 or lid 304 or receiver 112 or any combination thereof) includes some electronics functionality beyond just that associated with their respective sensor systems. This is particularly advantageous for modular smart cases in accordance with the present disclosure.

Furthermore, in some embodiments, a photodiode-based sensor system includes a photodiode array that has more elements than the number of reservoirs on the blister card for which it is intended. As discussed below, a sensor system having higher sensor density than the reservoirs can, for example, provide more detection data per reservoir, enable the use of the same smart case with blister cards having different sizes and/or formats, and more. For smart cases intended to be operative for a set of blister cards having different configurations, it is preferable that the density of photodiodes of a photodiode-based sensor system be equal to or greater than the highest reservoir density of any of the set of blister cards.

FIG. 6B depicts detection module 600 with the addition of optional cover 606.

Cover 606 is a thin decorative layer configured to hide sensor system 502. Cover 606 includes apertures 608, which are formed in the layer to enable light to pass through it and be received by photodiodes 408. In the depicted example, apertures 608 are through holes; however, a wide range of apertures are within the scope of the present disclosure, such as transparent regions that function as windows, etc. In some embodiments, apertures 608 include one or more optical elements (e.g., lenses, diffraction gratings, blazed gratings, etc.) for facilitating the detection of light at photodiodes 408.

It should be noted that the material and structure of cover 606 are based on the sensing technology used in sensor system 502. For example, for aesthetics it is often desirable to hide sensor system 502; therefore, in such instances, optically opaque material is desirable. When sensor system 502 employs capacitive sensing, however, the material of cover 606 must be electrically insulating and thin, and the cover would not typically include apertures 608.

As will be appreciated by one of ordinary skill in the art, smart medicine cases (or other smart packages) known in the prior art are configured for operation with only one particular blister card configuration and will not function for blister cards having different configurations. For the purposes of this Specification, including the appended claims, the “configuration” of a blister card is defined as the size and shape of the blister card, as a whole, and its blister-card parameters, which include the number, size, shape, and inter-reservoir spacing between its reservoirs. It is an aspect of the present disclosure, however, that a smart case can be configured to accept any of a set of blister cards having different configurations by employing an appropriate adapter, where necessary, for each blister card of the set.

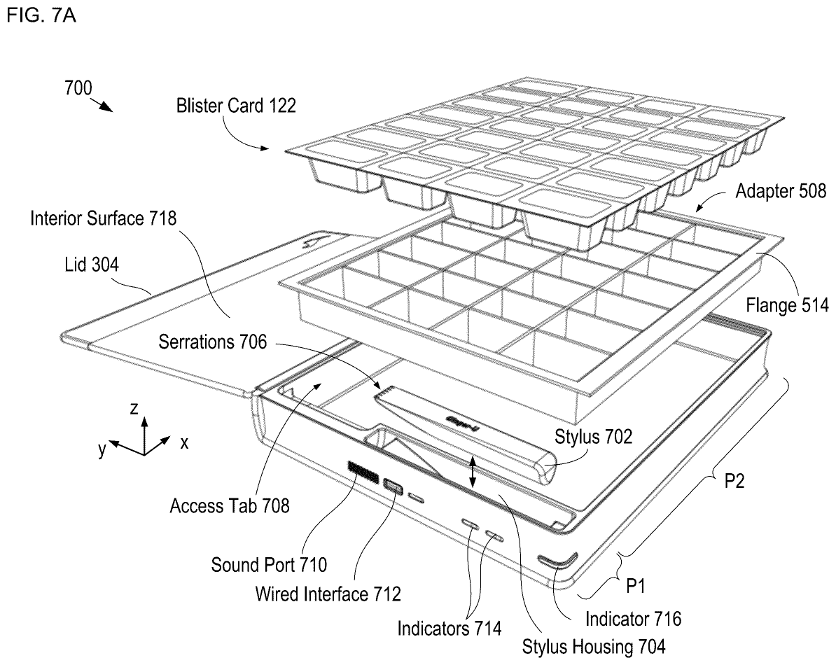

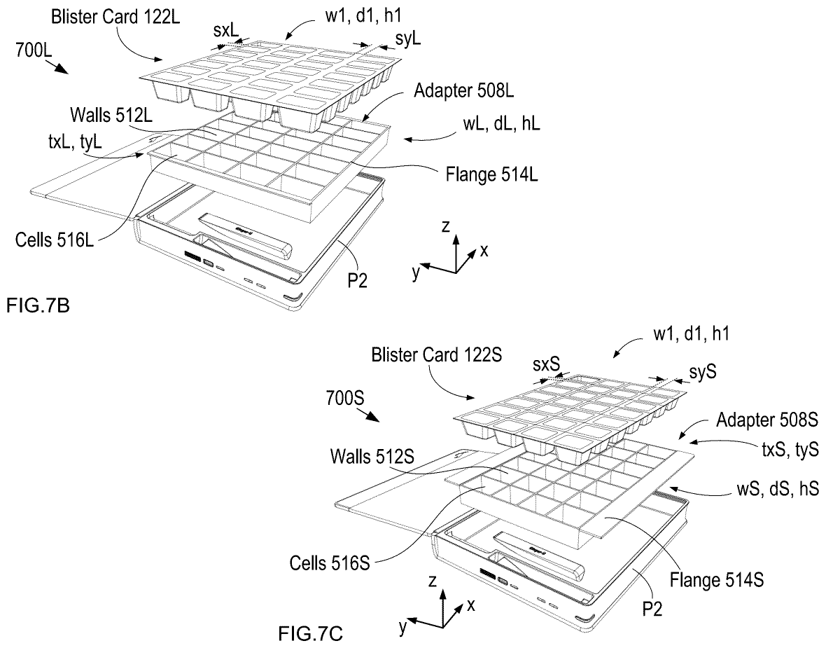

FIG. 7A depicts a schematic drawing of an exploded perspective view of the salient features of a reconfigurable smart case in accordance with the present disclosure. Case 700 is analogous to detection module 500; however, case 700 includes optional stylus 702, stylus housing 704, access tab 708, and more, all of which are located in body portion P1. In the example depicted in FIG. 7A, case 700 is configured to monitor that state of blister card 122, as discussed above.

Stylus 702 is designed for piercing dispensing regions 212 and scooping out content 208 from reservoirs 206. Although this function can often be done by the user using a finger or another object (e.g., fork, spoon, etc.), stylus 702 can provide significant advantages. For example, stylus 702 includes optional serrations 706 to facilitate puncture and removal of lidding film 204 in dispensing regions 212.

In the depicted example, body portion P1 also includes access tab 708 to facilitate removal of adapter 508. In some embodiments, adapter 508 includes an opposing recess in flange 514 to further facilitate removal of blister card 122.

FIG. 7A also depicts additional exemplary user interfaces, such as sound port 710, wired interface 712, and visual indicators 714. Non-limiting examples of user interfaces suitable for use in smart cases in accordance with the present disclosure are described in detail in the '874, '121, '779, and '021 applications. It should be noted that the interfaces depicted, and their locations, are merely exemplary and many different interfaces, at any suitable locations, can be included in a smart case without departing from the scope of the present disclosure.

Sound port 710 includes perforations and/or holes that allow sound to travel into and out of case 700. They are used in conjunction with microphones, speakers, sirens, buzzers, etc. for verbal interaction with the user, sound alerts and any other acoustic interfacing.

Wired interface port 712 is a conventional connection (e.g., USB, Micro USB, etc.) for enabling wired two-way communication and/or charging of an energy storage unit.

Indicators 714 are display elements (e.g., light-emitting diodes, incandescent lamps, liquid-crystal elements, etc.) for providing visual alerts and/or status indications to the user (e.g., visual dosing time reminder, the case is functioning correctly, energy storage unit is charging/charged, present adherence status, an issue must be addressed, etc.).

Indicator 716 is a visual display element that is configured to flash to gain the attention of the user. Indicator 716 is optionally included to assist a user who is hearing impaired in recognizing that interaction with case 700 is needed (e.g., a dosing window is happening or imminent, a dose has been missed, a dose has been improperly dispensed, etc.).

In some embodiments, one or more user interfaces are disposed on interior surface 718 of lid 304. Such user interfaces can include, for example, one or more displays, touch switches, mirrors, tabs/holders (e.g., for holding papers, labels, tablets, a smart phone, and the like), etc. to implement desired functionalities and features.

FIGS. 7B-C depict schematic drawings of exploded perspective views of smart case 700 as configured for use with blister cards that are larger and smaller, respectively, than blister card 122.

Case 700L represents smart case 700 as configured to accept and monitor blister card 122L. Case 700L includes case 700 and adapter 508L.

Blister card 122L has the same outer dimensions (i.e., overall size and shape) as blister card 122; however, at least one of the blister-card parameters (wL, dL, hL) of blister card 122L is different than those of blister card 122. Specifically, in the depicted example, blister card 122L includes the same reservoirs (i.e., reservoirs 206) as blister card 122; therefore, wL=w1, dL=d1, and hL=h1). However, the inter-reservoir spacings, sxL and syL, of the reservoirs is greater along each of the x- and y-dimensions than the inter-reservoir spacings of blister card 122. As a result, blister card 122L is characterized by a blister region 218L (not shown), which has a larger perimeter than blister region 218.

Adapter 508L includes flange 514L, which has the same outer perimeter (OP1) as adapter 508, thereby enabling adapter 508L to seat securely in body portion P2. Since the inner dimensions of flange 514L are configured to accept blister card 122L, flange 514L has an inner perimeter IPL (not shown) that is larger than that of flange 514. As a result, flange 514L is narrower than flange 514—in fact, it is non-existent along two of the outer edges of adapter 508L.

The dimensions and spacing of walls 512 of adapter 508L are selected based on the parameters of blister card 122L. Since reservoirs 206 are characterized by a larger inter-reservoir spacing in blister card 122L, walls 512L are thicker.

In similar fashion, case 700S includes case 700 and adapter 508S, where adapter 508S is configured to receive blister card 122S. Blister card 122S also has the same outer dimensions (i.e., overall size and shape) as blister card 122; however, at least one of the blister-card parameters (wS, dS, hS) of blister card 122S is different than those of each of blister cards 122 and 122L. Specifically, in the depicted example, blister card 122S includes the same reservoirs (i.e., reservoirs 206) as blister card 122; therefore, wS=w1, dS=d1, and hS=h1). however, the inter-reservoir spacings, sxS and syS, of the reservoirs is smaller along each of the x- and y-dimensions than the inter-reservoir spacings of blister card 122. As a result, blister card 122S is characterized by a blister region 218S (not shown), which has a smaller perimeter than blister region 218.

As a result, adapter 508S includes flange 514S, which has the same outer dimensions as adapter 508, thus enabling it to seat securely in body portion P2. The inner perimeter IPS (not shown) of flange 514S is configured to accept blister card 122S, thus it is smaller than that of flange 514 and flange 514S is wider than flange 514.

It should be noted that an adapter such as adapter 508S can be used to accommodate any blister card whose outer dimensions are no larger than those of blister card 122.

Since reservoirs 206 are characterized by a smaller inter-reservoir spacing in blister card 122S, walls 512S are thinner than walls 512.

Since adapters can be made rather inexpensively (e.g., with plastic injection molding), they provide an inexpensive way to accommodate a myriad of blister cards in the same smart case.

It should be noted that the examples provided above merely represent a few of a wide range of approaches for enabling a smart case to accommodate a wide range of blister card sizes and/or reservoir geometries within the scope of the present disclosure.

To accommodate blister cards having different configurations of reservoirs, a case must include a sensor system that is capable of monitoring the contents of each individual reservoir regardless of its position within the blister card. Sensor systems having a one-to-one correspondence to the distribution of reservoirs in a given blister card, for example, are typically not suitable for use with any blister card other than the type for which it is designed.

As noted briefly above, in some embodiments, a photodiode-based sensor system preferably includes a plurality of photodiodes that has a density greater than the highest reservoir density of any blister card for which the sensor system is operative. Specifically, the number and density of the photodiodes is preferably chosen such that there exists at least one photodiode within the optically isolated reservoir locations for each blister card of the set of blister cards with which the smart case is intended to operate. There is, however, a special condition that allows using the same number of photodiodes as reservoirs for different blister cards. For this special condition, the different blister cards must have: (1) the same number of reservoirs; and (2) a common area of overlap between their respective blister cards. For a set of such blister cards, a single arrangement of photodiodes having a one-to-one correspondence with the reservoirs could be provided such that each photodiode resides in the overlap area of a corresponding reservoir of each of the different blister cards.

In some embodiments, a smart case employs a sensor system that includes a focal plane array that can receive light and substantially image all of the reservoirs on a blister card, regardless of their location. Such a sensor system would be able to monitor virtually any reservoir arrangement.

It should be noted that a similar strategy can be used with sensor systems based on sensing technologies other than optical sensing, such as resistance sensing, impedance sensing, capacitive sensing, acoustic sensing, tactile sensing, etc. described in detail in the '874, '121, '779, and '021 applications. In fact, a highly dense sensing system can enable identification of the geometry of an unknown blister card inserted into a smart case.

Furthermore, artificial intelligence and machine learning can be used to determine the geometry and content of a blister/blister card, thereby allowing identification of an unknown blister card and its features, as well as those of an adapter loaded into the smart case.

It should be noted that when a dispensing region is pierced to enable removal of the contents of a reservoir, a downward force is typically exerted on the blister card. In some embodiments, therefore, adapter 508 includes features that function as detents to hold blister card 122 in place. As noted above, one or more surfaces of walls 512 can also be provided with a “tacky” surface that mitigates slippage of the blister card relative to the adapter.

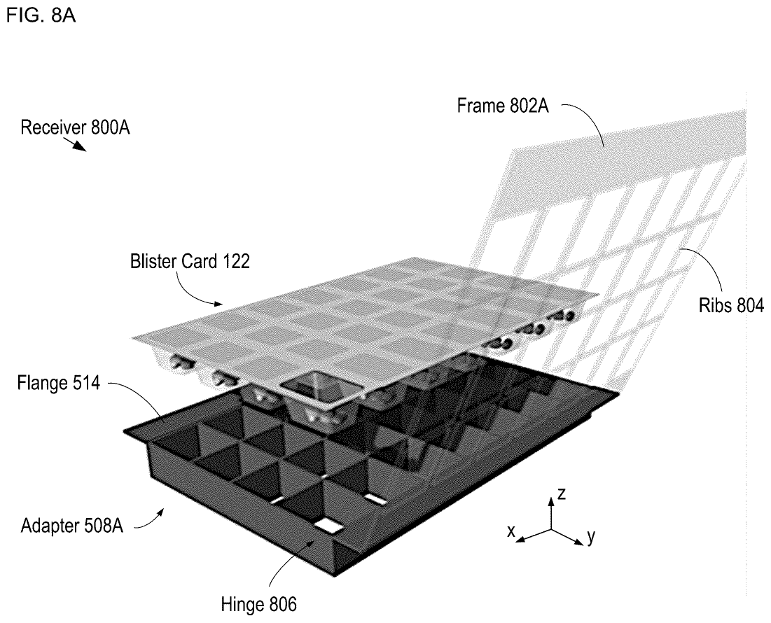

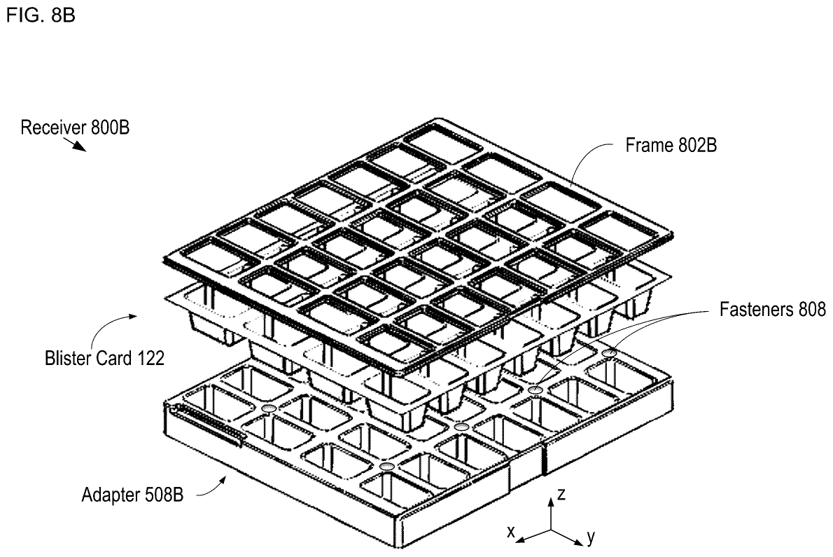

FIGS. 8A-B depict exploded schematic perspective drawings of two alternative receivers in accordance with the present disclosure. Receiver 800A includes seat 510 (not shown), adapter 508A, and frame 802A.

Frame 802A includes ribs 804, which are configured to lie between reservoirs 206 of blister card 122 when the frame is latched in place.

Frame 802A is attached to adapter 508A by hinge 806, which is located along the edge of the adapter where it meets frame 802A. Frame 802A also includes a conventional latch (not shown) configured to attach to flange 514 and secure frame 802A over blister card 122 to hold the blister card in place.

Receiver 800B is analogous to receiver 800A; however, in receiver 800B, frame 802B is magnetically attached to adapter 508B instead of via a hinge.

Each of frame 802B and adapter 508B includes a fastener 808 that is configured to hold the frame in place once it is placed over the blister card, which locks the blister card in place as well. In the depicted example, fastener 808 is a permanent magnet; however, other conventional fasteners (e.g., Velcro, mechanical latches, electromagnetic fasteners, etc.) can be used as fastener 808 without departing from the scope of the present invention.

It should be noted that some blister cards are designed to make them more child proof by using a multi-layer lidding film that is difficult to rupture or tear. Typically, the layers include a metal foil (e.g., aluminum) on the inside, paper on the outside, and a thin polymer layer sandwiched in between the metal and paper. In such blister card, peeling the lidding film off is a central step to accessing the content of a reservoir. Unfortunately, this peeling action applies an upward force on the blister card; however, when frames 802A and 802B are latched, the frame keeps the blister card in place.

Accordingly, embodiments in accordance with the present disclosure are suitable for use with such “peel-away” blister cards, even if a user elects to remove the blister card from the seat, tear off a dose (i.e., one or more reservoirs) along the card's perforation lines and place the card back into the seat. It should be noted that if the user is expected to always remove the peel-away card as just described, the seat need not include a frame, simplifying card removal and re-insertion of the card.

As long as a blister card is properly located in the receiver of a smart case in accordance with the present disclosure, the blister card can be interrogated for reservoirs removed from the card, empty reservoirs, partially empty reservoirs and untouched reservoirs. It should be noted that, for a smart case comprising a photodiode-based sensing system, a reservoir region removed from a blister card results in more light being incident on its respective photodiode or photodiodes than an empty blister remaining on the card.

One skilled in the art will appreciate that a reservoir can be considered a special shape of a bottle, vessel, vial, tub, tube, pouch, can, cartridge, and the like. Accordingly, the application of smart cases in accordance with the present disclosure is not limited to only blister-card formats.

Furthermore, the type of material contained in a content compartment can dictate the preferred type of sensing technology employed in a smart case. For example, a content compartment might contain loose pills, liquids, gels, syrups, suspensions, powders, pastes, creams, strips, vapor, spray, and the like, that is at least partially optically transparent. As a result, a sensing technology other than optical detection might be dictated. In some instances, a capacitive sensing scheme (including electrical impedance tomography) would be preferred if, for example, the content were electrically insulating. If the content is electrically conductive, electrical resistance or impedance sensing (including electrical resistance or impedance tomography) might be preferred. In some instances, acoustic sensing might be preferred.

It is to be understood that the disclosure teaches just some exemplary embodiments and that many variations of the invention can easily be devised by those skilled in the art after reading this disclosure and that the scope of the present invention is to be determined by the following claims.

1-24. (canceled)

25. A smart case (100) for monitoring a state of any blister card (122) of a set of blister cards, wherein the smart case includes:
a housing (102) comprising a body (302) having a first body portion (P1) and a second body portion (P2);
an electronics module (104) that is located in the first body portion; and
a detection module (106) that is located in the second body portion, wherein the detection module comprises:
a sensor system (114) that is configured to monitor a state of any blister card of the set thereof, wherein each blister card comprises a forming film (202), a lidding film (204), and a plurality of blisters (210) having perimeters (216) that collectively define a blister region (218), and wherein the forming film, lidding film and plurality of blisters collectively define a plurality of reservoirs for holding content;
a set of adapters (518), each adapter of the set thereof being dimensioned and arranged to removably locate a different blister card of the set thereof, wherein each adapter includes an annular element having:
(i) an outer perimeter (OP) that enables the adapter to seat securely within the second body portion; and
(ii) an opening (522) having an inner perimeter (IP), wherein the opening is configured to mitigate a lateral movement of its respective blister card when it is located in the adapter;
wherein each adapter of the set thereof has a different inner perimeter.

26. The smart case of claim 25 further comprising a set of frames (314), each frame of the set thereof being configured to attach to a different adapter of the set thereof, wherein each frame of the set thereof has at least one opening for exposing at least one reservoir of the plurality of reservoirs of its respective blister card, and wherein each frame and corresponding adapter are collectively configured to securely hold their respective blister card such that it is operatively coupled with the sensor system.

27. The smart case of claim 26 wherein the at least one opening of a first frame of the set thereof is a central opening that is configured to expose all of the reservoirs of its respective blister card.

28. The smart case of claim 26 wherein the at least one opening of a first frame of the set thereof includes a plurality of openings, each opening of the plurality thereof configured to expose a different reservoir of the plurality of reservoirs of its respective blister card.

29. The smart case of claim 25 wherein each adapter of the set thereof includes a recess (524) having a depth equal to the combined thickness of the forming film and lidding film of its respective blister card.

30. The smart case of claim 25 further comprising:
a lid (304); and
a latch (326);
wherein the latch has a first state that secures the lid and the body in a closed configuration and a second state that enables motion of the lid relative to the body, wherein the latch is an electrically-actuated latch that is configured to enable the second state only in response to a command from the electronics module.

31. The smart case of claim 30, wherein the smart case is configured to:
(1) identify an appropriate dosing window for each blister card of the plurality thereof;
(2) identify an authorized user; and
(3) issue the command only upon identification of an authorized user and during the appropriate dosing window.

32. The smart case of claim 25, wherein the smart case is configured to (1) identify one or more dosing windows for each blister card of the plurality thereof and (2) issue an alert upon detection of at least one event selected from the group consisting of:
(i) removal of content from one than one reservoir of the plurality thereof during one dosing window;
(ii) removal of a blister card from the smart case; and
(iii) removal of content from at least one reservoir of the plurality thereof during a time period not within an identified dosing window.

33. The smart case of claim 25 further comprising an access tab (708) for facilitating removal of any adapter of the set thereof from the second body portion.

34. The smart case of claim 25 wherein each adapter of the set thereof is configured to facilitate removal of its respective blister card of the set thereof.

35. The smart case of claim 25 further comprising a stylus (702) that is operative for piercing the lidding film of at least one blister card of the set thereof.

36. The smart case of claim 35, wherein the stylus (702) includes at least one serration (706).

37. A smart case (100) for monitoring a state of any blister card of a set of blister cards, wherein the smart case includes:
a housing (102) having a first body portion (P1) and a second body portion (P2);
an electronics module (104) that is located in the first body portion; and
a detection module (106) that is located in the second body portion, wherein the detection module comprises:
a sensor system (114) that is configured to monitor a state of any blister card of the set thereof, wherein each blister card comprises a forming film (202), a lidding film (204), and a plurality of blisters (210) having perimeters (216) that collectively define a blister region (218), and wherein the forming film, lidding film and plurality of blisters collectively define a plurality of reservoirs for holding content; and
a receiver that includes:
(a) a seat (312);
(b) at least one frame (314), each frame having at least one opening; and
(c) a set of adapters (518), each adapter of the set thereof being dimensioned and arranged to removably secure a different blister card of the set thereof, wherein each adapter includes an annular element having:
(i) an outer perimeter (OP) that enables the adapter to seat securely within the second body portion; and
(ii) an opening (522) having an inner perimeter (IP), wherein the opening is configured to mitigate a lateral movement of its respective blister card when it is located in the adapter;
wherein each adapter of the set thereof has a different inner perimeter, and wherein each adapter includes a recess (524) having a depth equal to the combined thickness of the forming film and lidding film of its respective blister card.

38. The smart case of claim 37 wherein the at least one frame includes a first frame having an annular element having a central opening that is configured to expose all of the reservoirs of any blister card of the plurality thereof.

39. The smart case of claim 37 wherein the at least one frame includes a plurality of frames (802), each frame of the plurality thereof being configured to attach to a different adapter of the set thereof to enable the frame and adapter to collectively securely hold their respective blister card such that it is operatively coupled with the sensor system, wherein a first frame of the plurality thereof includes an annular element and a plurality of ribs (804) that collectively define a plurality of openings, each opening of the plurality thereof being configured to expose a different reservoir of the plurality of reservoirs of its respective blister card of the set thereof.

40. The smart case of claim 37 further comprising:
a lid (304) and
a latch (326);
wherein the latch has a first state that secures the lid and the body in a closed configuration and a second state that enables motion of the lid relative to the body, wherein the latch is an electrically-actuated latch that is configured to enable the second state only in response to a command from the electronics module.

41. The smart case of claim 40, wherein the smart case is configured to:
(1) identify an appropriate dosing window for each blister card of the plurality thereof;
(2) identify an authorized user; and
(3) issue the command only upon identification of an authorized user and during the appropriate dosing window.

42. The smart case of claim 37, wherein the smart case is configured to (1) identify one or more dosing windows for each blister card of the plurality thereof and (2) issue an alert upon detection of at least one event selected from the group consisting of:
(i) removal of content from one than one reservoir of the plurality thereof during one dosing window;
(ii) removal of a blister card from the smart case; and
(iii) removal of content from at least one reservoir of the plurality thereof during a time period not within an identified dosing window.

43. The smart case of claim 37 further comprising an access tab (708) for facilitating removal of any adapter of the set thereof from the second body portion.

44. The smart case of claim 37 wherein each adapter of the set thereof is configured to facilitate removal of its respective blister card of the set thereof.

45. The smart case of claim 37 further comprising a stylus (702) that is operative for piercing the lidding film of at least one blister card of the set thereof.

46. The smart case of claim 45, wherein the stylus (702) includes at least one serration (706).
